(19) United States
(12) Patent Application Publication
(10) Pub. No.: US 2025/0372245 A1
(43) Pub. Date: Dec. 4, 2025

(54) DATA ACQUISITION CIRCUIT AND BIOLOGICAL SENSOR

(71) Applicants: Nitto Denko Corporation, Ibaraki-shi, Osaka (JP); SPChange, LLC., Yokohama-shi, Kanagawa (JP)

(72) Inventors: Ryoma YOSHIOKA, Ibaraki-shi, Osaka (JP); Takatsugu KAMATA, Yokohama-shi, Kanagawa (JP); Yusaku HIRAI, Yokohama-shi, Kanagawa (JP); Masayuki UEDA, Yokohama-shi, Kanagawa (JP)

(21) Appl. No.: 19/104,800

(22) PCT Filed: Aug. 23, 2023
(86) PCT No.: PCT/JP2023/030277

§ 371 Date: Feb. 19, 2025

(30) Foreign Application Priority Data
Aug. 25, 2022 (JP) 2022-133918

(51) Int. Cl. G16H 40/63; H03M 1/06; H03M 1/18

(52) U.S. Cl. CPC G16H 40/63; H03M 1/0626; H03M 1/18

(57) ABSTRACT

A data acquisition circuit includes a plurality of signal processors including a first analog chopper circuit, an amplifier, a second analog chopper circuit, and an analog filter that are coupled in series, respectively. The data acquisition circuit during a first mode converts analog signals from analog filters into a serial signal and converts the serial signal into a digital signal, and filters the digital signal after converting the digital signal into parallel signals. The data acquisition circuit during a second mode converts the analog signals from the amplifiers into the serial signal and converts the serial signal into the digital signal, outputs the digital signal to the digital chopper circuit, and filters the digital signal after converting the digital signal from the digital chopper circuit into parallel signals. Thus, biological information in the plurality of frequency bands can be appropriately acquired.

### TECHNICAL FIELD

The present invention relates to data acquisition circuits and biological sensors.

### BACKGROUND ART

For example, a biological sensor that acquires biological information from a living body includes an analog front end that amplifies a weak analog signal acquired as the biological information and converts the amplified analog signal into a digital signal (for example, refer to Patent Document 1). A chopper amplifier that removes flicker noise (1/f noise) generated when the analog signal is amplified is known (for example, refer to Patent Document 2). An oversampling data conversion circuit is known in which an analog chopper circuit is connected to an input of a sigma-delta modulator, and a digital filter and a digital chopper circuit are connected in sequence to an output of the sigma-delta modulator (for example, refer to Patent Document 3).

### PRIOR ART DOCUMENTS

### Patent Documents

Patent Document 1: Japanese Laid-Open Patent Publication No. 2020-163128
Patent Document 2: Japanese Laid-Open Patent Publication No. 2011-19156
Patent Document 3: U.S. Pat. No. 8,633,843

### DISCLOSURE OF THE INVENTION

### Problem to be Solved by the Invention

Recently, in order to acquire a plurality of types of biological information by a single biological sensor, multi-channeling and broadbanding of the biological sensor are required. However, a biological sensor and a data acquisition circuit mounted on the biological sensor, which satisfy such requirements, have not yet been proposed.

The present invention is conceived in view of the above, and one object of the present invention is to provide a data acquisition circuit and a biological sensor capable of appropriately acquiring biological information in a plurality of frequency bands.

### Means of Solving the Problem

A data acquisition circuit according to an embodiment of the present invention is characterized in that there are provided a plurality of signal processors including a first analog chopper circuit, an amplifier, a second analog chopper circuit, and an analog filter coupled in series, respectively, and configured to receive an analog signal by the first analog chopper circuit; a first converter configured to convert analog signals output from a plurality of analog filters into a serial signal during a first mode, and to convert the analog signals output from a plurality of amplifiers into a serial signal during a second mode; an analog-to-digital converter configured to convert the analog signals, converted into the serial signal by the first converter, into a digital signal; a digital chopper circuit coupled to an output of the analog-to-digital converter; a second converter configured to convert the digital signal output from the analog-to-digital converter into parallel digital signals during the first mode, and to convert the digital signal output from the digital chopper circuit into the parallel digital signals during the second mode; and a plurality of digital filters configured to filter the parallel digital signals converted by the second converter.

### Effects of the Invention

According to the disclosed technique, it is possible to provide a data acquisition circuit and a biological sensor capable of appropriately acquiring biological information in a plurality of frequency bands.

### BRIEF DESCRIPTION OF THE DRAWINGS

FIG. 1 is an overall configuration diagram [FIG. 1] illustrating an example of a biological sensor including a data acquisition circuit according to an embodiment.

FIG. 2 is a circuit block diagram illustrating an example of a circuit state of the data acquisition circuit of FIG. 1.

FIG. 3 is a circuit block diagram illustrating another example of the circuit state of the data acquisition circuit of FIG. 1.

FIG. 4 is a circuit diagram illustrating an example of chopper circuits illustrated in FIG. 2 and FIG. 3.

FIG. 5 is a timing chart illustrating an example of an operation of the data acquisition circuit of FIG. 2 in a digital chopping mode.

FIG. 6 is a timing chart illustrating a continuation of the operation of FIG. 5.

FIG. 7 is an explanatory diagram illustrating an outline of operations in the digital chopping mode and an analog chopping mode of the data acquisition circuits of FIG. 2 and FIG. 3.

FIG. 8 is an explanatory diagram illustrating examples of spectrums of a digital signal before being demodulated by a chopper circuit DCP of FIG. 2 and a digital signal output from a lowpass filter LPF (D) in the digital chopping mode.

FIG. 9 is an explanatory diagram illustrating an example of a band of an input signal that can be acquired in a normal mode and a broadband mode.

### MODE OF CARRYING OUT THE INVENTION

Hereinafter, embodiments for carrying out the invention will be described with reference to the drawings. In the following description, the same symbol as a signal name is used for a signal line through which information such as a signal is transmitted. Further, the same symbol as a voltage name is used for a voltage line through which a voltage is transmitted. In the drawings, the same constituent elements are designated by the same reference numerals, and a redundant description thereof may be omitted.

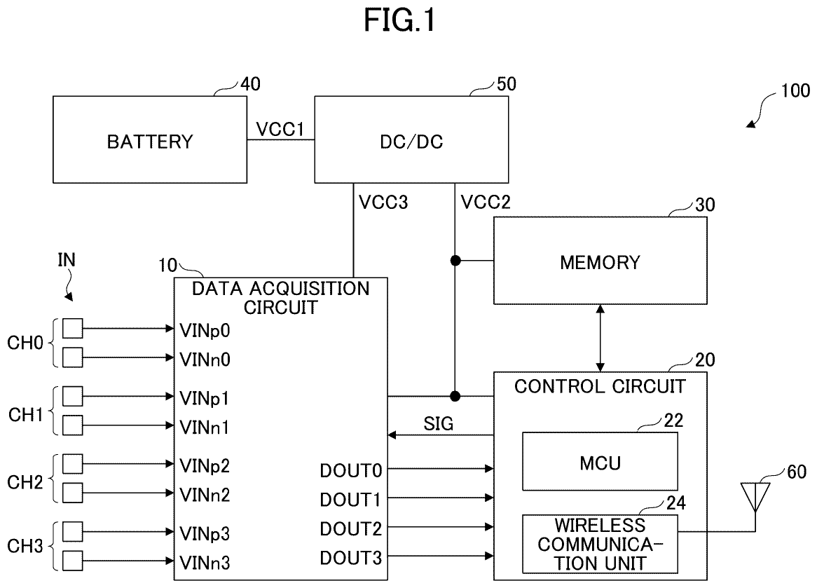

FIG. 1 is an overall configuration diagram illustrating an example of a biological sensor including a data acquisition circuit according to an embodiment. For example, a biological sensor 100 is a wearable device capable of acquiring a plurality of types of biological information from a living body.

The biological sensor 100 illustrated in FIG. 1 includes a data acquisition circuit 10, a control circuit 20, a memory 30, a battery 40, a DC/DC converter 50, and an antenna 60. Although not particularly limited, the data acquisition circuit 10, the control circuit 20, the memory 30, the battery 40, the DC/DC converter 50, and the antenna 60 are mounted on a circuit board implemented in the biological sensor 100.

For example, the data acquisition circuit 10 may be implemented in an Application Specific Integrated Circuit (ASIC) or a Field-Programmable Gate Array (FPGA). For example, the control circuit 20 may be implemented in a System on Chip (SoC) or a FPGA. Further, the data acquisition circuit 10 and the control circuit 20 may be implemented in a single FPGA.

For example, the memory 30 is an electrically rewritable nonvolatile memory, such as a flash memory, a Magnetoresistive Random Access Memory (MRAM), or the like. The biological sensor 100 does not require the battery 40 in a case where electric power is received from the outside. In addition, the biological sensor 100 does not require the battery 40 and the DC/DC converter 50 in a case where power supply voltages VCC2 and VCC3 are received from the outside.

The data acquisition circuit 10 receives differential input voltage signals VINp0/VINn0, VINp1/VINn1, VINp2/VINn2, and VINp3/VINn3 of four channels CH0 through CH3 by eight input terminals IN. Hereinafter, in a case where the channels CH0 through CH3 are described without making distinctions, these channels are also referred to as channels CH. In a case where the input voltage signals VINp0 through VINp3 are described without making distinctions, these input voltage signals are also referred to as input voltage signals VINp, and in a case where the input voltage signals VINn0 through VINn3 are described without making distinctions, these input voltage signals are also referred to as input voltage signals VINn. The number of channels of the data acquisition circuit 10 is not limited to four, as long as the number of channels is two or more.

For example, the input voltage signal VINp/VINn is a biological signal acquired from a living body via an electrode or various sensors, and represents biological information. The biological information is at least one of an electrocardiogram waveform, an electroencephalogram, a pulse, a blood pressure, an oxygen saturation, a body temperature, a heart sound, a breath sound, or the like. The biological sensor 100 may acquire the same type of biological information through the plurality of channels CH, or may acquire different types of biological information through the plurality of channels.

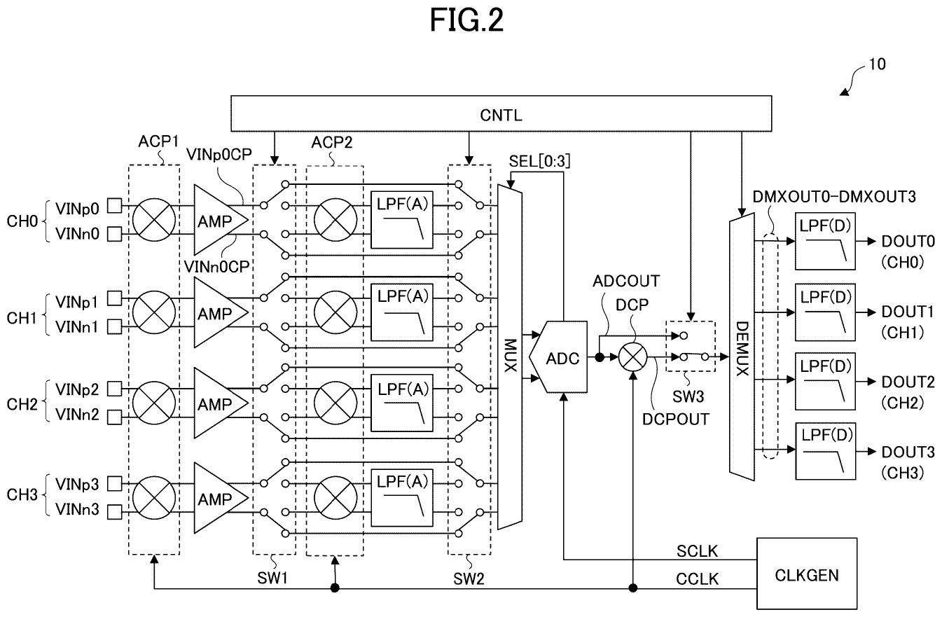

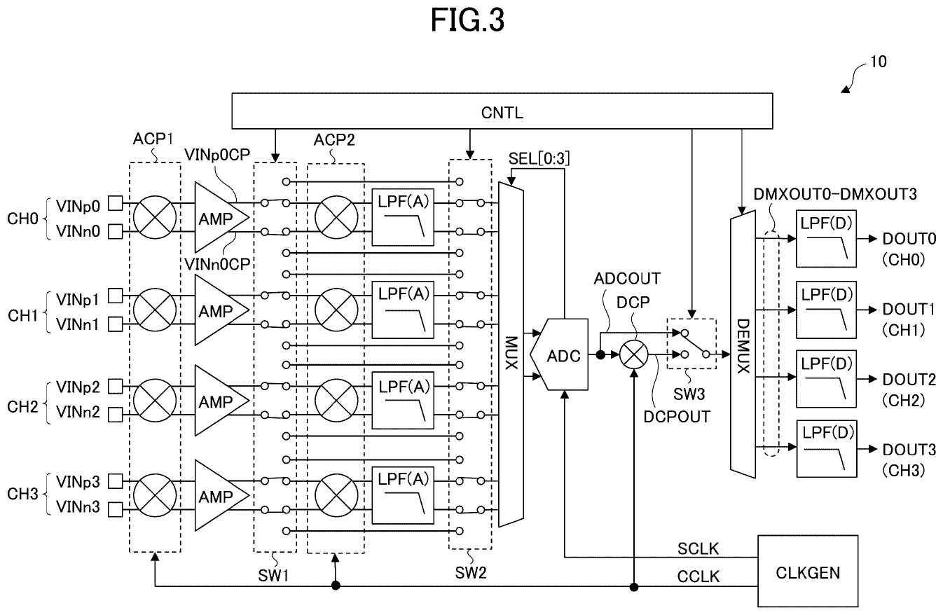

The data acquisition circuit 10 amplifies the received input voltage signals VINp/VINn, removes noise, and outputs digital output signals DOUT (DOUT0 through DOUT3) to the control circuit 20, for example. The digital output signal DOUT is a signal obtained by processing the input voltage signal VINp/VINn of the channel CH having the same numerical value affixed at the end thereof. Examples of the data acquisition circuit 10 are illustrated in FIG. 2 and FIG. 3.

The control circuit 20 includes a Micro Controller Unit (MCU) 22 and a wireless communication unit 24, for example. The MCU 22 controls the overall operation of the biological sensor 100 by executing a control program. For example, the MCU 22 may output an acquisition start instruction for starting acquisition of the biological information to the data acquisition circuit 10 via a signal line SIG, or may output an acquisition stop instruction for stopping the acquisition of the biological information to the data acquisition circuit 10 via the signal line SIG. Exchange of information between the control circuit 20 and the data acquisition circuit 10 may be performed via a serial interface, such as a Serial Peripheral Interface (SPI: registered trademark) or the like.

The MCU 22 receives the digital signals DOUT (biological information) output from the circuit 10, and writes the received biological information in the memory 30. The MCU 22 reads the biological information held in the memory 30, and outputs the biological information to the wireless communication unit 24. Although not particularly limited, the control circuit 20 and the memory 30 may be connected via a serial interface, such as the SPI or the like. The data acquisition circuit 10 may directly write the digital signals DOUT0 through DOUT3 into the memory 30. Further, the MCU 22 may receive the acquisition start instruction, the acquisition stop instruction, or the like with respect to the biological information from the outside via the wireless communication unit 24, and control the operation of the data acquisition circuit 10.

The wireless communication unit 24 communicates with an external device disposed outside the biological sensor 100, via the antenna 60, based on the control from the MCU 22. For example, the biological information transmitted from the wireless communication unit 24 to the external device may be the biological information held in the memory 30, or may be the biological information that is output from the data acquisition circuit 10 and before being written in the memory 30.

The battery 40 outputs a power supply voltage VCC1 to the DC/DC converter 50. The DC/DC converter 50 generates power supply voltages VCC2 and VCC3 using the power supply voltage VCC1. For example, the power supply voltage VCC2 is used as an operating power supply for the control circuit 20 and the memory 30, and is also used as an operating power supply for a digital circuit implemented in the data acquisition circuit 10. For example, the power supply voltage VCC3 is used as an operating power supply for an analog circuit implemented in the data acquisition circuit 10. A power supply voltage for data programming may be supplied to the memory 30. In this case, the DC/DC converter 50 may generate the power supply voltage for the programming.

FIG. 2 is a circuit block diagram illustrating an example of a circuit state of the data acquisition circuit 10 of FIG. 1. The data acquisition circuit 10 includes a chopper circuit ACP1, an amplifier AMP, a switch SW1, a chopper circuit ACP2, an analog lowpass filter LPF (A), a switch SW2, and a digital lowpass filter LPF (D), provided in correspondence with each of the channels CH0 through CH3. The data acquisition circuit 10 includes a multiplexer MUX, an analog-to-digital converter ADC, a chopper circuit DCP, a switch SW3, and a demultiplexer DEMUX, provided in common with respect to the channels CH0 through CH3. In addition, the data acquisition circuit 10 includes a control circuit CNTL that controls operations of the switches SW1 through SW3, and a clock generation circuit CLKGEN.

The chopper circuit ACP1, the amplifier AMP, the switch SW1, the chopper circuit ACP2, and the analog lowpass filter LPF (A) are an example of a signal processor. The switch SW1 is an example of a third selector. The switch SW2 and the analog-to-digital converter ADC are an example of a first converter. The switch SW3 and the demultiplexer DEMUX are an example of a second converter. The switch SW2 is an example of a first selector, and the switch SW3 is an example of a second selector.

Each chopper circuit ACP1 operates as a modulator that modulates the input voltage signal VINp/VINn (analog signal) in synchronism with a chopping clock CCLK, and outputs a differential voltage signal obtained by the modulation to the amplifier AMP. The chopper circuit ACP1 is an example of a first analog chopper circuit that modulates a voltage signal. The chopping clock CCLK is an example of a first clock.

Each amplifier AMP amplifies the differential voltage signal received from the chopper circuit ACP1, and outputs the amplified differential voltage signal to the switch SW1. For example, the amplifier AMP of the channel CH0 outputs a differential voltage signal VINP0CP/VINn0CP. The switch SW1 connects an output of the amplifier AMP to the switch SW2 or the chopper circuit ACP2, based on a control signal from the control circuit CNTL.

Each chopper circuit ACP2 operates as a demodulator that demodulates the (differential) voltage signal output from the amplifier AMP in synchronism with the chopping clock CCLK, and outputs the differential voltage signal obtained by the demodulation to the lowpass filter LPF (A). The chopper circuit ACP2 is an example of a second analog chopper circuit that demodulates the voltage signal.

Each lowpass filter LPF (A) removes a high-frequency component from the voltage signal received from the chopper circuit ACP2, and outputs the voltage signal removed of the high-frequency component to the switch SW2. The switch SW2 connects the output of the amplifier AMP or an output of the lowpass filter LPF (A) to the multiplexer MUX, based on a control signal from the control circuit CNTL.

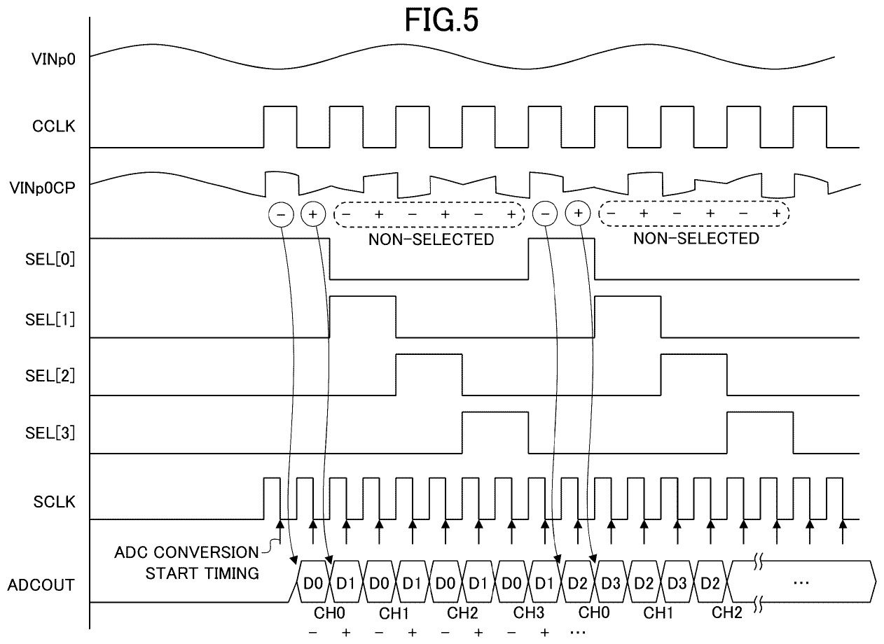

The multiplexer MUX sequentially selects the differential voltage signals of the channels CH0 through CH3 supplied via the switch SW2, according to a selection signal SEL[0:3] output from the analog-to-digital converter ADC. The multiplexer MUX outputs the selected differential voltage signal to the analog-to-digital converter ADC. The multiplexer MUX functions as a parallel-serial conversion circuit that converts voltage signals output in parallel from the plurality of lowpass filters LPF (A) or voltage signals output in parallel from the plurality of amplifiers AMP, into a serial signal. An example of the operation of the multiplexer MUX is illustrated in FIG. 5.

The multiplexer MUX converts the plurality of voltage signals supplied in parallel via the switch SW2 into the serial signal, and thus, the input voltage signals VINp/VINn of four channels can be converted into digital values by the single analog-to-digital converter ADC. Because it is unnecessary to provide a plurality of analog-to-digital converters ADC, even in a case where the plurality of input voltage signals VINp/VINn are to be processed, an increase in chip size of the data acquisition circuit 10 can be reduced.

The analog-to-digital converter ADC converts the differential voltage signals received from the multiplexer MUX into the digital values in synchronism with a sampling clock SCLK, and outputs the digital values as a digital signal ADCOUT. The sampling clock SCLK is an example of a second clock. The analog-to-digital converter ADC generates a 4-bit selection signal SEL[0:3] using the sampling clock SCLK, and outputs the generated selection signal SEL[0:3] to the multiplexer MUX. For example, a frequency of the sampling clock SCLK is two times a frequency of the chopping clock CCLK.

The analog-to-digital converter ADC sequentially generates the selection signal SEL[0:3] that is set to a high level during one period of the chopping clock CCLK, based on the sampling clock SCLK. That is, each bit of the selection signal SEL[0:3] is set to a high level during one period of the chopping clock CCLK, for every four cycles of the chopping clock CCLK. By generating the selection signal SEL[0:3] by the analog-to-digital converter ADC using the sampling clock SCLK, it becomes unnecessary to separately provide a circuit for generating the selection signal SEL[0:3]. As a result, it is possible to reduce a circuit scale of the data acquisition circuit 10.

The chopper circuit DCP operates as a demodulator that demodulates the digital signal ADCOUT received from the analog-to-digital convertor ADC, in synchronism with the chopping clock CCLK, and outputs a digital signal DCPOUT obtained by the demodulation to the switch SW3. The chopper circuit DCP is a digital chopper circuit that demodulates the digital signal.

The switch SW3 connects an output of the analog-to-digital converter ADC or an output of the chopper circuit DCP to the demultiplexer DEMUX, based on a control signal from the control circuit CNTL.

The demultiplexer DEMUX outputs digital signals of the channels CH0 through CH3 supplied via the switch SW3, as one of digital signals DMXOUT0 through DMXOUT3, based on a control signal received from the control circuit CNTL. Hereinafter, in a case where the digital signals DMXOUT0 through DMXOUT3 are described without making distinctions, these digital signals are referred to as digital signals DMXOUT. The digital signal DMXOUT is a signal corresponding to the channel CH having the same numerical value affixed at the end thereof.

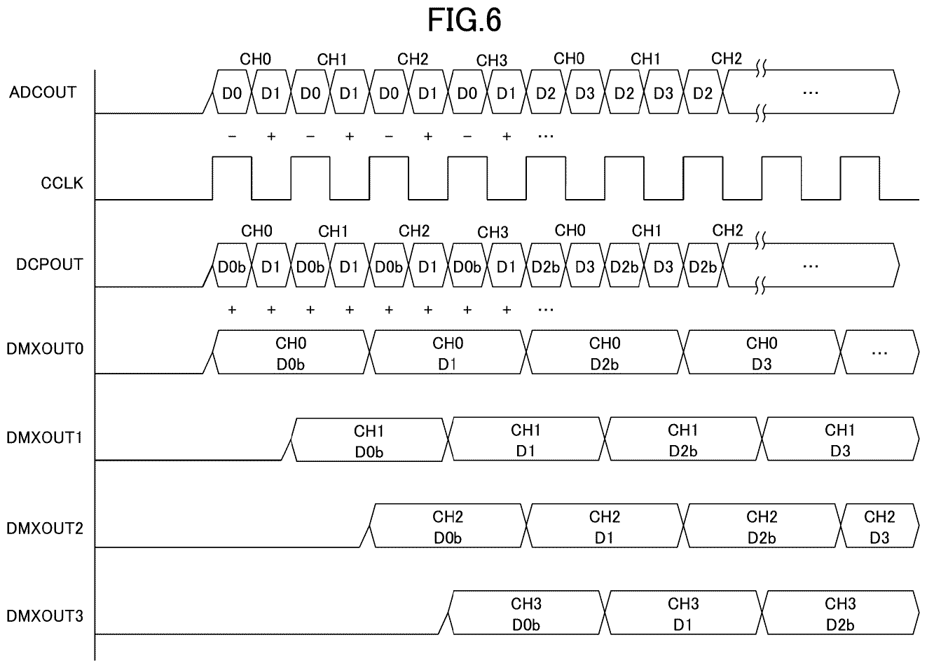

The demultiplexer DEMUX functions as a serial-parallel conversion circuit that converts the serial digital signal ADCOUT output from the analog-to-digital convertor ADC or the serial digital signal DCPOUT output from the chopper circuit DCP into the parallel digital signal DMXOUT. An example of the operation of the demultiplexer DEMUX is illustrated in FIG. 6.

Each of the lowpass filters LPF (D) performs filtering to remove a high-frequency component of the digital signal DMXOUT output from the demultiplexer DEMUX, and outputs a digital output signal DOUT (DOUT0 through DOUT3). Thus, the data acquisition circuit 10 can output four digital output signals DOUT corresponding to the input voltage signals VINp/VINn of the four channels, respectively.

The data acquisition circuit 10 operates the four lowpass filters LPF (D) in parallel, and generates the digital signals DOUT corresponding to the input voltage signals VINp/VINn of the four channels CH0 through CH3. Thus, even in a case where each lowpass filter LPF (D) performs a complex filtering process, the four digital signals DOUT0 through DOUT3 can be output in real time.

The clock generation circuit CLKGEN generates the chopping clock CCLK and the sampling clock SCLK.

States of the switches SW1, SW2, and SW3 in FIG. 2 indicate a state of a digital chopping mode in which the chopper circuits ACP2 and the lowpass filters LPF (A) are bypassed and the digital chopper circuit DCP is used as a demodulator. In the digital chopping mode, the switches SW1 and SW2 output the output of the amplifier AMP to the multiplexer MUX, thereby disconnecting the chopper circuits ACP2 and the lowpass filters LPF (A) from an operational path. The digital chopping mode is an example of a second mode.

In the digital chopping mode, the switch SW1 disconnects the output of the amplifier AMP from the chopper circuit ACP2, thereby reducing a charge and discharge current due to an operation of the chopper circuit ACP2. Accordingly, it possible to reduce a propagation delay time of the voltage signal VINP0CP/VINn0CP output from the amplifier AMP to the multiplexer MUX, for example, and to operate the data acquisition circuit 10 at a high speed. In addition, a power consumption of the data acquisition circuit 10 can be reduced.

In the digital chopping mode, the switch SW3 connects the output of the chopper circuit DCP to the demultiplexer DEMUX. That is, in the digital chopping mode, the chopper circuits ACP1, the amplifiers AMP, and the chopper circuit DCP function as a so-called chopping amplifier, and 1/f noise generated in the amplifiers AMP is modulated by the chopper circuit DCP and removed by the lowpass filters LPF (D).

For example, the data acquisition circuit 10 can acquire biological information processed at a relatively high sampling rate, such as a heart sound, a breath sound, or the like in the digital chopping mode. Hereinafter, the digital chopping mode is also referred to as a broadband mode.

The switch SW1 may be omitted in the data acquisition circuit 10. In this case, the output of the amplifier AMP is always connected to the chopper circuit ACP2 and the switch SW2. In the case where the switch SW1 is not provided, the supply of the chopping clock CCLK to the chopper circuit ACP2 may be stopped and a chopping clock terminal CCLK of the chopper circuit ACP2 may be fixed to a low level during the digital chopping mode. In addition, the data acquisition circuit 10 may include a switch between the analog-to-digital converter ADC and the chopper circuit DCP. In this case, the switch connects the analog-to-digital converter ADC and the chopper circuit DCP during the digital chopping mode, and disconnects the analog-to-digital converter ADC from the chopper circuit DCP during an analog chopping mode.

The demultiplexer DEMUX and the lowpass filters LPF (D) may be disposed outside the data acquisition circuit 10 (for example, inside the control circuit 20 of FIG. 1). In this case, the chip size of the data acquisition circuit 10 can be reduced. Further, because the output (amounting to four channels) of the switch SW3 can be output as a 1-bit digital output signal DOUT, a number of digital output signal lines between the data acquisition circuit 10 and the control circuit 20 can be reduced. As a result, it is possible to reduce a size of a substrate on which the data acquisition circuit 10, the control circuit 20, or the like are implemented.

FIG. 3 is a circuit block diagram illustrating another example of the circuit state of the data acquisition circuit 10 of FIG. 1. FIG. 3 is the same as FIG. 2, except for the different states of the switches SW1 through SW3. The states of the switches SW1 through SW3 in FIG. 3 indicate a state of the analog chopping mode in which the chopper circuits ACP2 and the lowpass filters LPF (A) are used and the chopper circuit DCP is bypassed. The analog chopping mode is an example of a first mode.

In the analog chopping mode, the switch SW1 connects the output of the amplifier AMP to the chopper circuit ACP2, and the switch SW2 connects the output of the lowpass filter LPF (A) to the multiplexer MUX. Moreover, in the analog chopping mode, the chopper circuit DCP is disconnected from the operational path.

That is, in the analog chopping mode, the chopper circuits ACP1, the amplifiers AMP, and the chopper circuits ACP2 operate as a chopping amplifier, and the 1/f noise generated in the amplifier AMP is modulated by the chopper circuits ACP2 and removed by the lowpass filters LPF (A).

For example, the data acquisition circuit 10 can acquire the biological information processed at a relatively low sampling rate, such as an electrocardiogram waveform, an electroencephalogram, a pulse, a blood pressure, an oxygen saturation level, a body temperature, or the like in the analog chopping mode. Hereinafter, the analog chopping mode is also referred to as a normal mode.

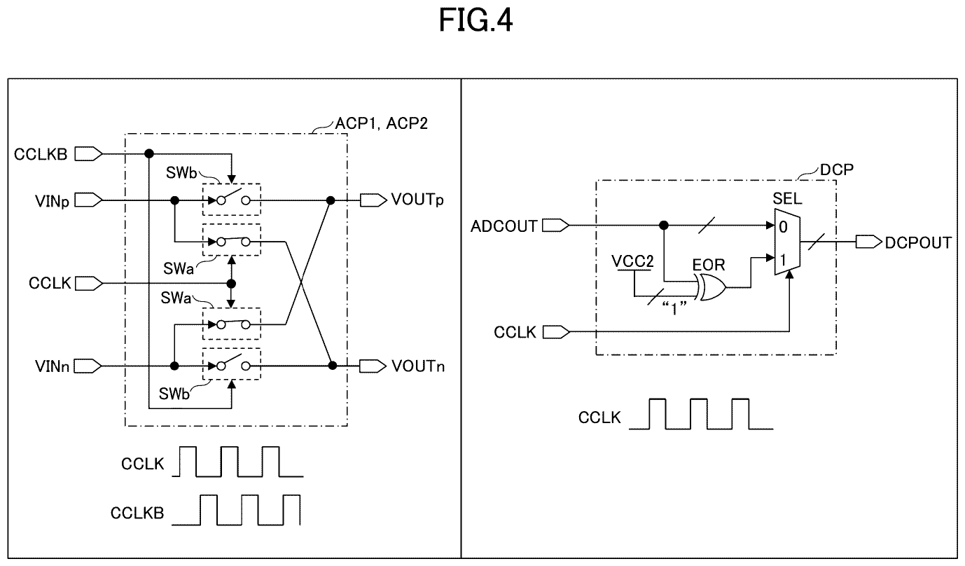

FIG. 4 is a circuit diagram illustrating an example of the chopper circuits ACP1, ACP2, and DCP of FIG. 2 and FIG. 3. The chopper circuits ACP1 and ACP2 are identical circuits. Each of the chopper circuits ACP1 and ACP2 includes a pair of switches SWa and a pair of switches SWb. As described above, in the case where the data acquisition circuit 10 does not include the switch SW1, the supply of the chopping clocks CCLK and CCLKB to the chopper circuit ACP2 may be stopped during the digital chopping mode. In this case, the chopping clocks CCLK and CCLKB are fixed to the low level and the high level, respectively.

The switch SWa is turned on during a high-level period of the chopping clock CCLK, outputs an input voltage signal VINp as an output voltage VOUTn, and outputs an input voltage signal VINn as an output voltage VOUTp. The switch SWb is turned on during a high-level period of the chopping clock CCLKB, outputs the input voltage signal VINp as the output voltage VOUTp, and outputs the input voltage signal VINn as the output voltage VOUTn. The chopping clock CCLKB is a clock having a phase opposite to that of the chopping clock CCLK, and having the high-level period that does not overlap the high-level period of the chopping clock CCLK.

Each of the chopper circuits ACP1 and ACP2 switches on and off states of the switches SWa and SWb once per period of the chopping clock CCLK, and inverts polarities of output signals VOUTP and VOUTN. The operation of each of the chopper circuits ACP1 and ACP2 mathematically corresponds to a process of multiplying a rectangular wave to waveforms of the input voltage signals VINp and VINn.

The chopper circuit DCP includes an exclusive OR circuit EOR and a selector SEL. The exclusive OR circuit EOR receives a digital input signal DIN and a logical value “1”, and outputs a signal having an inverted logic of the digital input signal DIN to the selector SEL. The selector SEL outputs the digital input signal DIN as a digital output signal DOUT during a low-level period of the chopping clock CCLK.

The selector SEL outputs the signal having the inverted logic of the digital input signal DIN as the digital output signal DOUT during the high-level period of the chopping clock CCLK. Thus, the chopper circuit DCP outputs the digital output signal DOUT by inverting the logic of the digital input signal DIN with the period of the chopping clock CCLK.

FIG. 5 and FIG. 6 are timing diagrams illustrating an example of the operation of the data acquisition circuit 10 of FIG. 2 in the digital chopping mode. Although FIG. 5 illustrates the waveform of the input voltage signal VINp0 of the channel CH0, the data acquisition circuit 10 receives the differential input voltage signals VINp and VINn of all of the channels CH0 through CH3.

First, the chopper circuit ACP1 inverts the polarity of the differential input voltage signal VINp0/VINn0 during the high-level period of the chopping clock CCLK. The amplifier AMP amplifies the differential voltage signal having polarity thereof inverted during the high-level period of the chopping clock CCLK, and outputs a differential voltage signal VINP0CP/VINn0CP. In FIG. 5, the waveform of the voltage VINn0CP is omitted. A symbol “−” illustrated below the waveform of the voltage signal VINp0CP indicates that the polarity thereof has been inverted. A symbol “+” illustrated below the waveform of the voltage signal VINP0CP indicates that the polarity thereof has not been inverted.

The multiplexer MUX receives the voltage signal (for example, VINp0CP) output from the amplifier AMP via the switches SW1 and SW2. The multiplexer MUX sequentially selects the voltage output from the amplifier AMP of the channel CH corresponding to one of the 4-bit selection signals SEL[0:3] set to a high level for each cycle of the chopping clock CCLK.

The multiplexer MUX outputs the selected voltage signal to the analog-to-digital converter ADC. The input voltage signal VINp0 corresponding to the channel CH0 is selected during a high-level period of the selection signal SEL[0], and is not selected during a high-level period of the other selection signals SEL[1:3]. The multiplexer MUX may receive a 2-bit selection signal that repeats updating from 0 to 3, and decode the received selection signal to internally generate the selection signal SEL[0:3].

The analog-to-digital converter ADC sequentially converts the voltage signal received from the multiplexer MUX into the digital value ADCOUT in synchronism with the sampling clock SCLK. For example, a frequency of the sampling clock SCLK is two or more times the frequency of the chopping clock CCLK. For this reason, the analog-to-digital converter ADC samples a voltage signal having the polarity “−” and a voltage signal having the polarity “+” in one period of the chopping clock CCLK.

Thus, the analog-to-digital converter ADC can sequentially acquire the voltage signals VINP0CP/VINn0CP having the polarities “−” and “+” as the digital values ADCOUT (the data D0, D1, D2, and D3). In addition, because the bits of the selection signal SEL[0:3] are sequentially set to the high level, the analog-to-digital converter ADC can sequentially acquire the digital value ADCOUT (data D0, D1, D2, and D3) for each of the channels CH0 through CH3.

In FIG. 6, the chopper circuit DCP inverts the polarity of the digital value ADCOUT received from the analog-to-digital convertor ADC, and outputs the digital value having the inverted polarity as the digital value DCPOUT during the high-level period of the chopping clock CCLK. The chopper circuit DCP outputs the digital value ADCOUT received from the analog-to-digital convertor ADC as the digital value DCPOUT, without inverting the polarity of the digital value ADCOUT during the low-level period of the chopping clock CCLK. Data D0b and D2b illustrated in the digital signal DCPOUT indicate that the polarity is inverted.

The digital value ADCOUT received by the chopper circuit DCP during the high-level period of the chopping clock CCLK corresponds to the voltage signal VINP0CP/VINn0CP inverted of the polarity thereof and amplified by the chopper circuit ACP1. Thus, the digital value DCPOUT output from the chopper circuit DCP is returned (demodulated) to the polarity of the input voltage signal VINp/VINn supplied to the data acquisition circuit 10.

The demultiplexer DEMUX outputs the digital value DCPOUT received from the chopper circuit DCP to one of the four lowpass filters LPF (D) as a digital value DMXOUT (DMXOUT0 through DMXOUT3). For example, each lowpass filter LPF (D) receives the digital value DMXOUT for every four periods of the chopping clock CCLK. In addition, the digital values DMXOUT received by the four lowpass filters LPF (D) are out of phase by one cycle of the chopping clock CCLK, respectively.

Accordingly, the digital values ADCOUT output in series from the analog-to-digital converter ADC can be supplied to the four lowpass filters LPF (D), as parallel digital values DMXOUT corresponding to the channels CH0 through CH3, respectively.

Thereafter, each of the lowpass filters LPF (D) illustrated in FIG. 2 removes a high-frequency component from the received digital value DMXOUT, and outputs the digital value removed of the high-frequency component as the digital output signal DOUT (DOUT0 through DOUT3) illustrated in FIG. 2. For example, the digital output signals DOUT (DOUT0 through DOUT3) are output to the control circuit 20 of FIG. 1.

The operation in the analog chopping mode is the same as that in FIG. 5 and FIG. 6, except that the multiplexer MUX selects the output of the lowpass filter LPF (A) in FIG. 5 and the demultiplexer DEMUX outputs the digital signal ADCOUT as the digital signal DMXOUT in FIG. 6.

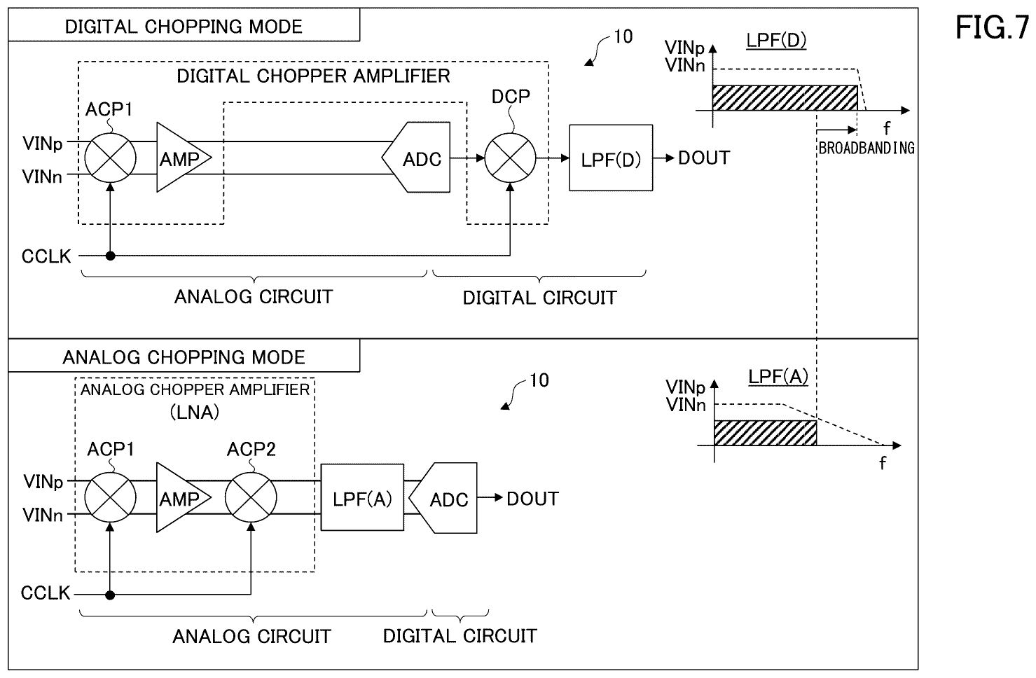

FIG. 7 is an explanatory diagram illustrating an outline of the operations in the digital chopping mode and the analog chopping mode of the data acquisition circuit 10 of FIG. 2 and FIG. 3. In the digital chopping mode, the analog chopper circuits ACP1 for modulation and the amplifiers AMP, and the digital chopper circuit DCP for demodulation connected to the output of the analog-to-digital convertor ADC constitute a digital chopper amplifier.

In the digital chopper amplifier, the modulated analog signals amplified by the amplifiers AMP are converted into digital signals, and thereafter demodulated by the digital chopper circuit DCP. Further, the 1/f noise generated by the amplification in the amplifiers AMP is moved to a high-frequency side by the digital chopper circuit DCP for demodulation, and is removed by the digital lowpass filters LPF (D). Passbands of the digital lowpass filters LPF (D) are broader than passbands of the analog lowpass filters LPF (A), and a boundary of the band for blocking the signal is steep. For this reason, the input voltage signals VINp/VINn in the broadband removed of the noise can be acquired as the biological information.

On the other hand, in the analog chopping mode, the analog chopper circuits ACP1 for modulation, the amplifiers AMP, and the analog chopper circuits ACP2 for demodulation constitute an analog chopper amplifier (Low Noise Amplifier: LNA). The 1/f noise generated by the amplification in the amplifiers AMP is moved to a high-frequency region by the analog chopper circuits ACP2, and is removed by the analog lowpass filters LPF (A). However, the passbands of the analog lowpass filters LPF (A) are narrower than the passbands of the digital lowpass filters LPF (D), and the boundary of the band for blocking the signal is gradual, and thus, the passbands of the input voltage signals VINp/VINn become limited.

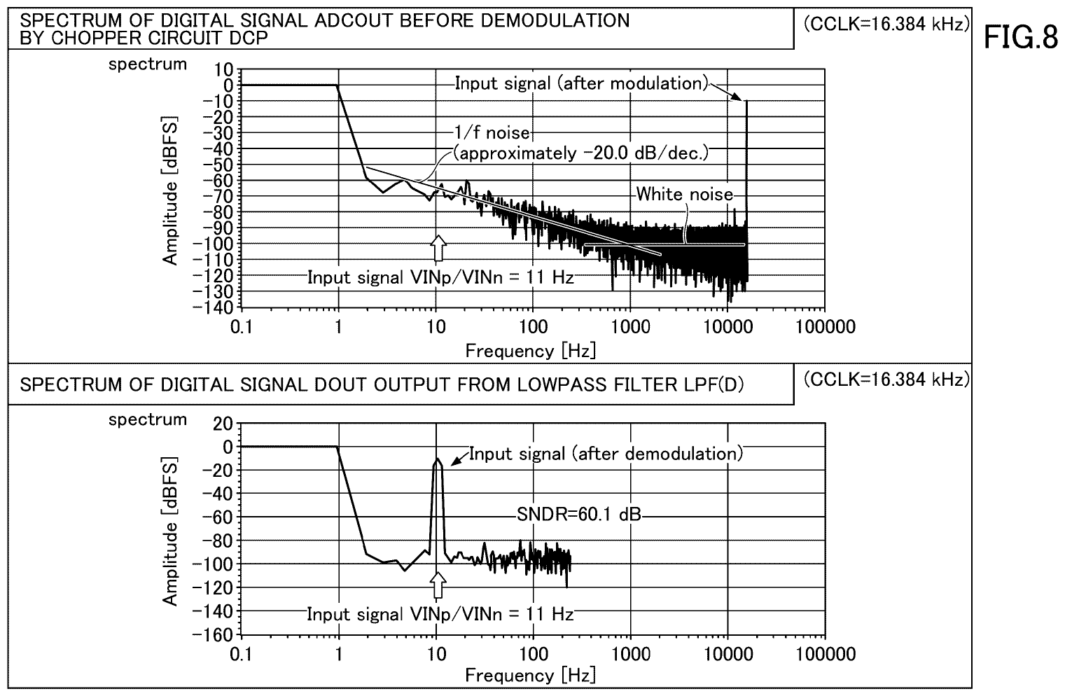

FIG. 8 is an explanatory diagram illustrating examples of spectrums of the digital signal before being demodulated by the chopper circuit DCP of FIG. 2 and the digital signal output from the lowpass filter LPF (D) in the digital chopping mode. An upper part of FIG. 8 illustrates the spectrum of the digital signal ADCOUT output from the analog-to-digital converter ADC of FIG. 2 and supplied to the chopper circuit DCP. A lower part of FIG. 8 illustrates the spectrum of the digital signal DOUT output from the digital lowpass filter LPF (D). Although not particularly limited, a frequency of the input voltage signal VINp/VINn in FIG. 8 is 11 Hz.

In the spectrum of the digital signal ADCOUT, the input voltage signal VINp/VINn indicated by the digital signal ADCOUT is shifted by the modulation of the chopper circuit ACP1 to 16.384 kHz which is the same as the frequency of the chopping clock CCLK. The spectrum of the digital signal ADCOUT includes the 1/f noise and white noise.

On the other hand, in the spectrum of the digital signal DOUT, the input voltage signal VINp/VINn indicated by the digital signal DOUT is returned to the original frequency of 11 Hz by the demodulation of the chopper circuit DCP. In addition, the 1/f noise moved to the high-frequency band by the demodulation of the chopper circuit DCP is removed by the lowpass filter LPF (D). For example, a SNDR (signal to noise and distortion ratio) of the digital signal DOUT is 60.1 dB, which is good.

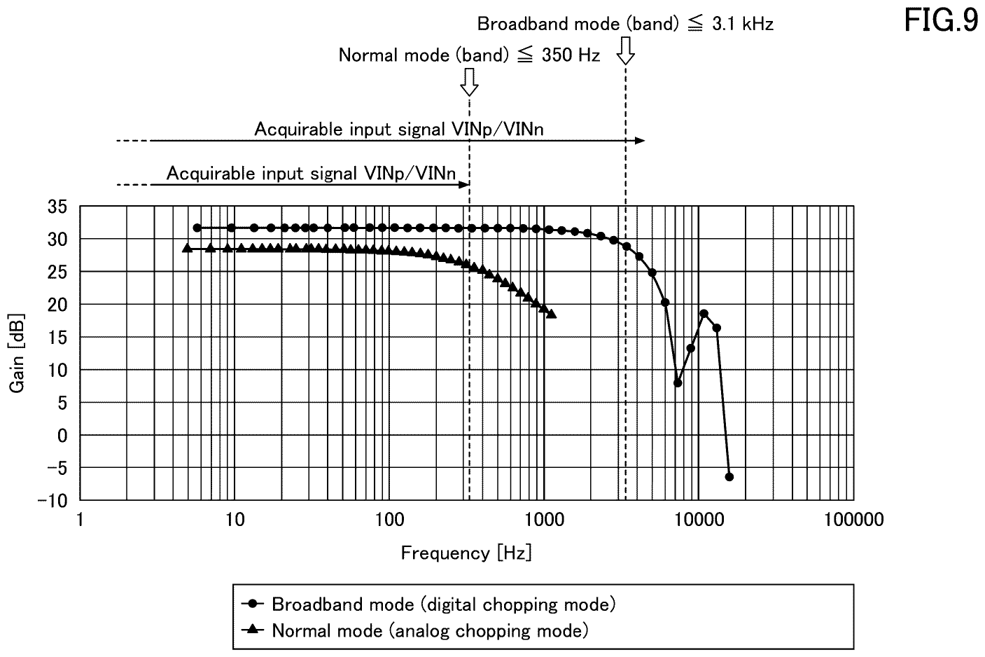

FIG. 9 is an explanatory diagram illustrating an example of the band of the input voltage signal VINp/VINn that can be acquired in the normal mode (analog chopping mode) and the broadband mode (digital chopping mode). A cutoff frequency of the input voltage signal VINp/VINn on the high band side is 350 Hz in the normal mode, and 3.1 kH in the broadband mode.

Accordingly, the data acquisition circuit 10 can be operated in an appropriate mode between the analog chopping mode and the digital chopping mode according to the band of the input voltage signal VINp/VINn. For example, in the normal mode, the white noise of the amplifier AMP or external noise can be reduced in some cases when compared to the broadband mode. In this case, in an application for acquiring only a narrowband signal, such as an electrocardiogram signal or the like, it is better to acquire the input voltage signal in the normal mode. The cutoff frequency of the input voltage signal VINp/VINn on the low band side is independent of the mode, and is 0.1 Hz or lower, for example.

As described above, according to the present embodiment, in the data acquisition circuit 10 and the biological sensor 100 capable of acquiring the plurality of input voltage signals VINp/VINn by time division multiplexing, it is possible to switch the band in which the input voltage signals VINp/VINn can be acquired, according to the operation mode. Hence, the biological information to be processed at a relatively low sampling rate, such as the electrocardiogram waveform, the electroencephalogram, the pulse, the blood pressure, the oxygen saturation, the body temperature, or the like, and the biological information to be processed at a relatively high sampling rate, such as the heart sound, the breath sound, or the like, for example, can be processed by the single biological sensor 100.

That is, it is possible to provide the data acquisition circuit 10 and the biological sensor 100 capable of appropriately acquiring the biological information in a plurality of frequency bands. In other words, it is possible to provide a multi-type biological sensor 100 having both the function of an electrocardiograph and the function of a phonocardiograph, for example. In this case, it is unnecessary to provide a data acquisition circuit for the electrocardiograph and a data acquisition circuit for the phonocardiograph in the biological sensor 100. As a result, the cost of the multi-type biological sensor 100 can be reduced, and a size of the multi-type biological sensor 100 can be reduced.

For example, by alternately switching between the analog chopping mode and the digital chopping mode, it is possible to acquire the biological information in the low frequency band and the biological information in the high frequency band in time division.

By using the multiplexer MUX and the demultiplexer DEMUX, the analog-to-digital converter ADC and the chopper circuit DCP can be used in common with respect to the plurality of differential input voltage signals VINp/VINn. Thus, the circuit scale of the data acquisition circuit 10 can be reduced compared to the case where the analog-to-digital converter ADC and the chopper circuit DCP are provided with respect to each of the plurality of differential input voltage signals VINp/VINn.

During the digital chopping mode, by disconnecting the output of the amplifier AMP from the chopper circuit ACP2 by the switch SW1, and stopping the supply of the chopping clock CCLK to the chopping clock CCLK, it is possible to reduce the power consumption of the data acquisition circuit 10.

By generating the selection signal SEL[0:3] by the analog-to-digital converter ADC using the sampling clock SCLK, it becomes unnecessary to separately provide a circuit for generating the selection signal SEL[0:3]. In this case, it possible to reduce the circuit scale of the data acquisition circuit 10.

By operating the four lowpass filters LPF (D) in parallel, even in the case where the complex filtering process is performed by each of the lowpass filters LPF (D), it is possible to output the four digital signals DOUT0 through DOUT3 in real time.

During the digital chopping mode, by disconnecting the output of the amplifier AMP from the chopper circuit ACP2 by the switch SW1, it is possible to reduce a charge and discharge current due to the operation of the chopper circuit ACP2. In this case, it possible to reduce a propagation delay time of the voltage signal VINP0CP/VINn0CP output from the amplifier AMP to the multiplexer MUX, for example, and to operate the data acquisition circuit 10 at a high speed. In addition, the power consumption of the data acquisition circuit 10 can be reduced.

Although the present invention is described based on the embodiments, the present invention is not limited to the specifically disclosed embodiments, and modifications can be made without departing from the subject matter of the present invention.

For example, the data acquisition circuit 10 is not limited to the application to the biological sensor 100, and may be implemented in a sensor that detects a signal other than the biological signal, such as temperature, light, pressure, geomagnetism, or the like. Further, the data acquisition circuit 10 may process a single-phase input signal, for example, instead of the differential input voltage signal VINp/VINn.

Aspects of the present invention include the following, for example.
<1> A data acquisition circuit characterized in that there are provided:
a plurality of signal processors including a first analog chopper circuit, an amplifier, a second analog chopper circuit, and an analog filter coupled in series, respectively, and configured to receive an analog signal by the first analog chopper circuit;
a first converter configured to convert analog signals output from a plurality of analog filters into a serial signal during a first mode, and to convert the analog signals output from a plurality of amplifiers into a serial signal during a second mode;
an analog-to-digital converter configured to convert the analog signals, converted into the serial signal by the first converter, into a digital signal;
a digital chopper circuit coupled to an output of the analog-to-digital converter;
a second converter configured to convert the digital signal output from the analog-to-digital converter into parallel digital signals during the first mode, and to convert the digital signal output from the digital chopper circuit into the parallel digital signals during the second mode; and
a plurality of digital filters configured to filter the parallel digital signals converted by the second converter.
<2> The data acquisition circuit according to clause <1>, characterized in that:
the plurality of amplifiers operate in parallel,
the first analog chopper circuit, the second analog chopper circuit, and the digital chopper circuit operate in synchronism with a common first clock, and
the first converter includes:
   a first selector configured to select the analog signals output from the plurality of analog filters during the first mode, and to select the analog signals output from the plurality of amplifiers during the second mode, and
   a multiplexer configured to sequentially select the analog signals selected by the first selector for each period of the first clock.
<3> The data acquisition circuit according to clause <2>, characterized in that the analog-to-digital converter performs:
sampling of the analog signals in synchronism with a second clock having a frequency that is two or more times a frequency of the first clock, and
generating a selection signal for causing the multiplexer to sequentially select one of the analog signals, using the second clock.
<4> The data acquisition circuit according to <2> or <3>, characterized in that:
the second converter includes:
   a second selector configured to select the digital signal output from the analog-to-digital converter during the first mode, and to select the digital signal output from the digital chopper circuit during the second mode, and
   a demultiplexer configured to output the digital signal selected by the second selector in a sequence from a plurality of outputs for each period of the first clock, and
the plurality of digital filters are coupled to an output of the demultiplexer, respectively.
<5> The data acquisition circuit according to any one of <1> to <4>, characterized in that there is further provided:
a third selector configured to couple the output of the amplifier to the second analog chopper circuit in the first mode, and to couple the output of the amplifier to the first converter in the second mode.
<6> The data acquisition circuit according to any one of <1> to <5>, characterized in that the plurality of analog filters and the plurality of digital filters are lowpass filters.
<7> A biological sensor characterized in that there are provided a data acquisition circuit configured to acquire biological information, and a control circuit configured to control an operation of the data acquisition circuit,
wherein the data acquisition circuit includes:
   a plurality of signal processors including a first analog chopper circuit, an amplifier, a second analog chopper circuit, and an analog filter coupled in series, respectively, and configured to receive an analog signal by the first analog chopper circuit;
   a first converter configured to convert analog signals output from a plurality of analog filters into a serial signal during a first mode, and to convert the analog signals output from a plurality of amplifiers into a serial signal during a second mode;
   an analog-to-digital converter configured to convert the analog signals, converted into the serial signal by the first converter, into a digital signal;
   a digital chopper circuit coupled to an output of the analog-to-digital converter;
   a second converter configured to convert the digital signal output from the analog-to-digital converter into parallel digital signals during the first mode, and to convert the digital signal output from the digital chopper circuit into the parallel digital signals during the second mode; and
   a plurality of digital filters configured to filter the parallel digital signals converted by the second converter.

This application is based upon and claims priority to Japanese Patent Application No. 2022-133918, filed on Aug. 25, 2022 before the Japan Patent Office, the entire contents of which are incorporated herein by reference.

### DESCRIPTION OF REFERENCE NUMERALS

10: Data acquisition circuit
20: Control circuit
22: MCU
24: Wireless communication unit
30: Memory
40: Battery
50: DC/DC converter
60: Antenna
100: Biological sensor
ACP1, ACP2: Chopper circuit
ADC: Analog-to-digital converter
ADCOUT: Digital signal
AMP: Amplifier
CCLK: Chopping clock
CH0-CH3: Channel
CLKGEN: Clock generation circuit
DCP: Chopper circuit
DCPOUT: Digital signal
DEMUX: Demultiplexer
DMXOUT0-DMXOUT3: Digital signal
DOUT0-DOUT3: Digital output signal
IN: Input terminal
LPF(A), LPF(D): Lowpass filter
MUX: Multiplexer
SCLK: Sampling clock
SW1-SW3: Switch
VINn0-VINn3: Input voltage signal
VINn0CP: Voltage signal
VINp0-VINp3: Input voltage signal
VINP0CP: Voltage signal

1. A data acquisition circuit comprising:
a plurality of signal processors including a first analog chopper circuit, an amplifier, a second analog chopper circuit, and an analog filter coupled in series, respectively, and configured to receive an analog signal by the first analog chopper circuit;
a first converter configured to convert analog signals output from a plurality of analog filters into a serial signal during a first mode, and to convert the analog signals output from a plurality of amplifiers into a serial signal during a second mode;
an analog-to-digital converter configured to convert the analog signals, converted into the serial signal by the first converter, into a digital signal;
a digital chopper circuit coupled to an output of the analog-to-digital converter;
a second converter configured to convert the digital signal output from the analog-to-digital converter into parallel digital signals during the first mode, and to convert the digital signal output from the digital chopper circuit into the parallel digital signals during the second mode; and
a plurality of digital filters configured to filter the parallel digital signals converted by the second converter.

2. The data acquisition circuit as claimed in claim 1, wherein:
the plurality of amplifiers operate in parallel,
the first analog chopper circuit, the second analog chopper circuit, and the digital chopper circuit operate in synchronism with a common first clock, and
the first converter includes:
a first selector configured to select the analog signals output from the plurality of analog filters during the first mode, and to select the analog signals output from the plurality of amplifiers during the second mode, and
a multiplexer configured to sequentially select the analog signals selected by the first selector for each period of the first clock.

3. The data acquisition circuit as claimed in claim 2, wherein the analog-to-digital converter performs:
sampling of the analog signals in synchronism with a second clock having a frequency that is two or more times a frequency of the first clock, and
generating a selection signal for causing the multiplexer to sequentially select one of the analog signals, using the second clock.

4. The data acquisition circuit as claimed in claim 2, wherein:
the second converter includes:
a second selector configured to select the digital signal output from the analog-to-digital converter during the first mode, and to select the digital signal output from the digital chopper circuit during the second mode, and
a demultiplexer configured to output the digital signal selected by the second selector in a sequence from a plurality of outputs for each period of the first clock, and
the plurality of digital filters are coupled to an output of the demultiplexer, respectively.

5. The data acquisition circuit as claimed in claim 1, further comprising:
a third selector configured to couple the output of the amplifier to the second analog chopper circuit in the first mode, and to couple the output of the amplifier to the first converter in the second mode.

6. The data acquisition circuit as claimed in claim 1, wherein the plurality of analog filters and the plurality of digital filters are lowpass filters.

7. A biological sensor comprising a data acquisition circuit configured to acquire biological information, and a control circuit configured to control an operation of the data acquisition circuit,
wherein the data acquisition circuit includes:
a plurality of signal processors including a first analog chopper circuit, an amplifier, a second analog chopper circuit, and an analog filter coupled in series, respectively, and configured to receive an analog signal by the first analog chopper circuit;
a first converter configured to convert analog signals output from a plurality of analog filters into a serial signal during a first mode, and to convert the analog signals output from a plurality of amplifiers into a serial signal during a second mode;
an analog-to-digital converter configured to convert the analog signals, converted into the serial signal by the first converter, into a digital signal;
a digital chopper circuit coupled to an output of the analog-to-digital converter;
a second converter configured to convert the digital signal output from the analog-to-digital converter into parallel digital signals during the first mode, and to convert the digital signal output from the digital chopper circuit into the parallel digital signals during the second mode; and
a plurality of digital filters configured to filter the parallel digital signals converted by the second converter.
